(19) United States
(12) Patent Application Publication
(10) Pub. No.: US 2023/0052667 A1
(43) Pub. Date: Feb. 16, 2023

(54) AGENT FOR MAINTAINING OR ENHANCING COLLAGEN PRODUCTION ABILITY

(71) Applicant: Shiseido Company, Ltd., Chuo-ku, Tokyo (JP)

(72) Inventors: Kazuyuki MIYAZAWA, Tokyo (JP); Renaud GILLET, Tokyo (JP); Bianca MCCARTHY, Tokyo (JP); Tetsuya KANEMARU, Tokyo (JP)

(73) Assignee: Shiseido Company, Ltd., Chuo-ku, Tokyo (JP)

(21) Appl. No.: 17/796,577

(22) PCT Filed: Jan. 29, 2021
(86) PCT No.: PCT/JP2021/003393

§ 371 Date: Jul. 29, 2022

(30) Foreign Application Priority Data
Jan. 31, 2020 (JP) 2020-015714

(51) Int. Cl. A61K 8/99; A61K 8/27; A61K 8/24; A61K 8/67

(52) U.S. Cl. CPC A61K 8/99; A61K 8/27; A61K 8/24; A61K 8/67

(57) ABSTRACT

A novel an agent for maintaining or promoting collagen productivity is provided with.

An agent for maintaining or promoting collagen productivity of cells, comprising a wavelength conversion substance as an active ingredient; a composition and a product comprising the agent for maintaining or promoting collagen productivity; and a method for maintaining or promoting collagen productivity by means thereof are provided with. The present invention can exhibit a desirable effect on skin by effectively making use of ultraviolet light to maintain or promoting collagen productivity in skin cells.

### FIELD

The present invention relates to an agent for maintaining or promoting collagen productivity in cells comprising a wavelength conversion substance, to a composition and product comprising the agent for maintaining or promoting collagen productivity in the cells, and to a method for maintaining or promoting collagen productivity in skin cells using the same.

### BACKGROUND

The skin is subjected to various physical an chemical stresses such as UV, dryness, cold air, heat, drugs in daily life. The harm to skin caused by ultraviolet light includes adverse effects such as skin cancer, photoaging, skin spots, wrinkles and inflammation, which are also undesirable from the viewpoint of health and beauty. While ultraviolet light is also utilized for sterilizing purposes, when considered in balance with the harm caused by ultraviolet light there is a greater need to defend against rather than to actively utilize ultraviolet light.

Many measures are therefore being taken to protect the skin from ultraviolet light. Such measures include the use of sunscreens, the implementation of indoor spaces for avoidance of sunlight, and the use of head coverings and clothing treated to block UV rays and films designed to block UV rays.

Paying an attention to wrinkles as the harmful influence by ultraviolet rays, two type of wrinkles are known, i.e., epidermal wrinkles and dermal wrinkles, among which dermal wrinkles are formed by the influence of ultraviolet rays contained in sunlight and aging. The formation mechanism of the dermal wrinkles includes decreased synthesis of the collagen from the dermis fibroblast due to the influence of ultraviolet ray and aging, as well as and decompose promotion of the collagen due to the increase of matrix metalloproteinase (MMP). Significant reductions in collagen fibers, the major matrix component of the dermis, are a major cause for reducing the skin thickness. Therefore, it is considered that maintaining the amount of collagen by promoting the collagen productivity is effective for preventing and improving wrinkles and sagging caused by, for example, ultraviolet irradiation.

### CITATION LIST

### Patent Literature

[PTL 1] Japanese Patent Publication No. 6424656
[PTL 2] Japanese Patent Publication No. 6361416
[PTL 3] International Patent Publication No. 2018/004006
[PTL 4] Japanese Unexamined Patent Publication No. 2018-131422
[PTL 5] Japanese Unexamined Patent Publication HEI No. 5-117127
[PTL 6] Japanese Patent Publication No. 4048420
[PTL 7] Japanese Patent Publication No. 4677250
[PTL 8] Japanese Patent Publication No. 3303942
[PTL 9] Japanese Unexamined Patent Publication No. 2017-88719
[PTL 10] International Patent Publication No. 2018/117117

### SUMMARY

### Technical Problem

It is an object of the present invention to provide an agent for maintaining or promoting collagen productivity in cells that utilizes ultraviolet light.

### Solution to Problem

The present inventors have conducted active research with the aim of allowing ultraviolet light to be effectively utilized on skin. As a result, an agent for promoting collagen productivity in skin cells has been devised that converts ultraviolet light wavelengths and activates skin cells.

The present application provides the present invention with the aspects set forth below.

(1) An agent for maintaining or promoting collagen productivity in cells comprising a wavelength conversion substance as an active ingredient,

wherein the wavelength conversion substance converts the wavelength of ultraviolet light contained in incident light to emit emission light having a wavelength longer than the wavelength of the ultraviolet light.

(2) The agent for maintaining or promoting collagen productivity according to (1), wherein the ultraviolet light has a peak wavelength between 200 nm and 400 nm.

(3) The agent for maintaining or promoting collagen productivity according to (1) or (2), wherein the emission light has a peak wavelength between 450 nm and 700 nm.

(4) The agent for maintaining or promoting collagen productivity cell activator according to any one of (1) to (3), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, and polyphenols; and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, and meta-aminophenol.

(4) The agent for maintaining or promoting collagen productivity according to (4), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.

(6) A composition comprising the agent for maintaining or promoting collagen productivity according to any one of (1) to (5).

(7) The composition according to (6), wherein the composition is a skin external composition for maintaining or promoting collagen productivity in cells under irradiation of light containing ultraviolet light.

(8) A cosmetic method for maintaining or promoting collagen productivity in the skin cells of a subject, comprising:

applying the composition according to (6) or (7) to skin of a subject, and

exposing the composition-applied skin with light containing ultraviolet light.

(9) A product comprising the agent for maintaining or promoting collagen productivity according to any one of (1) to (5).

(10) The product according to (9), wherein the product is for maintaining or promoting collagen productivity in skin cells under irradiation of light which contains ultraviolet light.

(11) A cosmetic method for maintaining or promoting collagen productivity in skin cells of a subject, comprising:

bringing light containing ultraviolet light through the product according to (9) or (10), and

exposing the skin of the subject to light passing through the product.

### Advantageous Effects of Invention

The present invention can exhibit a desirable effect on skin by maintaining or promoting collagen productivity in the skin cells while exposing to ultraviolet light. It is conventionally understood that ultraviolet light is undesirable for skin, in particular, that ultraviolet light lowers collagen productivity by skin cells as well as promotes the degradation of collagen, which results in wrinkle formation, and thus it has been common technical knowledge in the field to apply measures to maximally avoid exposing skin to ultraviolet light. It was highly surprising to find, then, that the present invention provides a desirable function, such as enhanced collagen productivity of the skin cells are maintained or promoted, while the skin is exposed to ultraviolet light. The invention provides novel uses of the aforementioned compounds that have conventionally been used primarily as dyes, pigments, ultraviolet scattering agents, ultraviolet absorbers, nutrients and antioxidants. The invention also helps to improve quality of life by providing a more positive feeling for persons who have attempted to avoid ultraviolet light as much as possible for beauty or health reasons when outdoors.

### BRIEF DESCRIPTION OF DRAWINGS

FIG. 1 shows an effect for maintaining or promoting the collagen productivity by using wavelength conversion substance under exposure to artificial sun light containing ultraviolet light. Item 1 corresponds to Sham, Item 2 corresponds to UV irradiation only, Item 3 corresponds to Zinc Oxide fluorescent material, Item 4 corresponds to magnesium titanate, and Item 5 corresponds to C-phycocyanin.

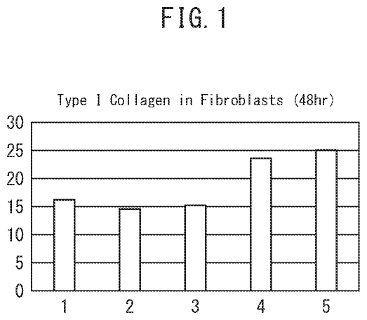

### DESCRIPTION OF EMBODIMENTS

The agent for maintaining or promoting collagen productivity of the invention comprises a wavelength conversion substance as an active ingredient. A wavelength conversion substance is a substance that converts the wavelength of ultraviolet light contained in incident light and emits emission light having a wavelength longer than the wavelength of the ultraviolet light.

The ultraviolet light may include UVA, UVB and UVC. According to one embodiment, the ultraviolet light is light with a peak wavelength of 200 nm to 400 nm. The ultraviolet light may also be included in incident light such as sunlight, for example. Alternatively, the incident light may be ultraviolet light, and artificially generated ultraviolet light may be used.

The emission light emitted by the wavelength conversion substance has a longer wavelength than ultraviolet light, with a peak wavelength of preferably 450 nm to 700 nm, more preferably 500 nm to 700 nm. The emission light may have one or more peaks at 450 nm, 450 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm or 700 nm, or in any range within these values, though without being restrictive, or it may be red light, orange light, green light or blue light. According to one embodiment, the wavelength conversion substance has its main wavelength at 450 nm to 700 nm, more preferably 500 nm to 700 nm, for light emitted upon excitation with excitation light of 200 nm to 400 nm.

Examples of wavelength conversion substances include the following components: phycobiliproteins such as allophycocyanin, C-phycocyanin, R-phycocyanin, phycoerythrocyanin, B-phycoerythrin, b-phycoerythrin, C-phycoerythrin and R-phycoerythrin; natural or synthetic components such as vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, polyphenols, dyes such as Red No. 401, Red No. 227, Red No. 504, Red No. 218, Orange No. 205 P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, meta-aminophenol, and phosphors obtained by fluorescent-doping of inorganic compounds, for example, blue phosphors comprising the amorphous silica particles mentioned in Japanese Patent No. 6424656, cerium and phosphorus and/or magnesium, and red phosphors comprising compounds obtained by europium activation of mixed crystals consisting of the alkaline earth metal sulfides described in Japanese Patent No. 6361416 combined with gallium compounds, the zinc oxide phosphors mentioned in International Patent Publication No. 2018/004006, the zinc oxide phosphors mentioned in Japanese Unexamined Patent Publication No. 2018-131422, and the inorganic phosphors mentioned in Japanese Unexamined Patent Publication HEI No. 5-117127. According to one embodiment, the inorganic phosphor is one or more phosphors selected from among phosphors obtained by doping zinc oxides represented by ZnO: Zn, Zn_{1+z}, ZnO₁₋ₓ with the sulfur-containing compounds mentioned in International Patent Publication No. 2018/004006, including sulfides and/or sulfates such as zinc sulfide or zinc sulfate, magnesium titanate phosphors obtained by doping magnesium titanates such as MgTiO₃ or Mg₂TiO₄ with manganese, and calcium phosphate phosphors obtained by doping calcium phosphates such as Ca(H₂PO₄)₂, CaHPO₄ or Ca₃(PO₄)₂ with cerium.

The wavelength conversion substance may be obtained by extraction from a natural source such as an animal, plant or algae, or it may be obtained by an artificial method such as chemical synthesis. For example, phycobiliproteins can be prepared by extraction from algae, including blue-green algae such as Spirulina (Spirulina platensis) or red algae such as porphyridiophylla (Porphyridium purpureum), by the method described in Japanese Patent No. 4048420, Japanese Patent No. 4677250 or Japanese Patent No. 3303942, for example. Zinc oxide phosphors can be produced by the method described in International Patent Publication No. 2018/004006, Japanese Unexamined Patent Publication No. 2018-131422 or Japanese Unexamined Patent Publication HEI No. 5-117127, for example. Magnesium titanate phosphors can be produced by the method described in Japanese Unexamined Patent Publication No. 2017-88719. Calcium phosphate phosphors can be produced by the method described in International Patent Publication No. 2018/117117.

So long as the wavelength conversion effect of the invention is not impaired, these wavelength conversion substances may be composed of, or may include, the components mentioned above, and they may be single components alone or combinations of more than one of the components. For example, the aforementioned phycobiliproteins or inorganic material phosphors may be mixed with other wavelength conversion substances such as B vitamins (vitamin B1, vitamin B2, vitamin B6 or vitamin B12) to exhibit synergistic effects. These components are merely examples, however, and any other substances that exhibit the wavelength conversion effect of the invention may be used.

The wavelength conversion substance content in the agent for maintaining or promoting collagen productivity, composition or product of the invention is not particularly restricted so long as the wavelength conversion effect of the invention is not impaired, and it may be appropriately determined for the type of wavelength conversion substance and the purpose of use of the agent for maintaining or promoting collagen productivity. It may be any content in the range of 0.01 to 99.99 wt % or 0.1% to 999 wt %, for example.

The agent for maintaining or promoting collagen productivity is effectively used for preventing aging of human skin, maintaining elasticity or tenseness of skin, preventing and treating arthritis, and early treatment of burns through maintaining or promoting good collagen productivity. It can be applied to a skin external preparation which displays the concept of physiological functions such as treatment, prevention, and improvement of the above symptoms and pathological conditions.

The measurement of the collagen productivity may be performed by a well-known method, and it is possible to measure the collagen productivity, but is not particularly limited to, by means of fluorescence which is emitted by 3,4-DHPAA binding to peptides having glycine at N-terminus, which is conducted in the collagen quantification kit of Cosmo Bio, as described in the Examples. The collagen constituting the skin includes type I, type III, type IV, type V, type XII, type XIV, and the like, and preferably refers to, but not particularly limited to, type I.

Any form of administration may be used for the agent for maintaining or promoting collagen productivity and composition of the invention, but a skin external preparation will often be preferred, such as a drug, quasi drug or cosmetic, for maintaining or promoting collagen productivity in skin cells along with exposing the skin to light containing ultraviolet light. When the agent for maintaining or promoting collagen productivity or composition of the invention is to be used as an external preparation for skin, the dosage form, coating method and number of doses may be determined as desired. For example, it may be applied onto skin in the form of cosmetic water or a spray, oil, cream, latex, gel, sunscreen or suntan lotion either periodically or irregularly, once or several times per day at morning, noon or evening, or before going out or engaging in outdoor activities, marine sports or skiing, for example, when exposure to sunlight is expected.

The agent for maintaining or promoting collagen productivity of the present invention and composition of the invention may also be used in combination with an additive such as an excipient, preservative, thickener, binder, disintegrator, dispersing agent, stabilizer, gelling agent, antioxidant, surfactant, preservative, oil, powder, water, alcohol, thickener, chelating agent, silicone, antioxidant, humectant, aromatic, drug component, antiseptic agent, pH adjustor or neutralizer, selected as necessary or desired. It may also be used in combination with other cell activators to increase the effect of the invention.

The present invention further provides products such as sun visors, caps, clothing, gloves, screen films, window sprays or creams, window materials or wall materials, for example, that comprise an agent for maintaining or promoting collagen productivity of the invention and are intended to maintain or promote collagen productivity of the skin cells along with exposing the skin to light containing ultraviolet light. The usage of additives in the products of the invention and the forms of the products may also be as desired.

The present invention further provides a method for producing the agent for maintaining or promoting collagen productivity, composition or product of the invention. A method for maintaining or promoting collagen productivity in skin cells of a subject is also provided, the method comprising application of an agent for maintaining or promoting collagen productivity or composition of the invention onto the skin of a subject and exposing the skin to light containing ultraviolet light after application of the agent for maintaining or promoting collagen productivityor composition; or passing light containing ultraviolet light through the product of the invention, and exposing the skin to the transmitted light; wherein the agent for maintaining or promoting collagen productivity, composition or product converts the wavelength of ultraviolet light in the incident light and emits emission light with a longer wavelength than the wavelength of the ultraviolet light, transmitting the ultraviolet light with a peak wavelength of preferably 200 nm to 400 nm as light with a peak wavelength of 450 nm to 700 nm, preferably 500 nm to 700 nm. The present invention further provides a method for promoting collagen production of skin cells of a subject, wherein the method comprises applying the agent for promoting collagen productivity or composition of the present invention to the skin of the subject, and exposing the skin after application of the agent for promoting collagen or composition to light containing ultraviolet light; or passing the product of the present invention to light containing ultraviolet light, and exposing the skin of the subject to the passing light; wherein the agent for maintaining or promoting collagen productivity, composition, and product convert the wavelength of the ultraviolet light contained in the incident light to emit emitted light having a wavelength longer than the wavelength of the ultraviolet light, and preferably changing ultraviolet light having a peak wavelength of 200 nm to 400 nm, to preferably 450 nm to 700 nm, more preferably 500 nm to 700 nm, and preferably containing magnesium titanate and/or C-phycocyanin. The method for maintaining or promoting the collagen productivity in skin cells of a subject is intended for cosmetic purposes and may not be used by physicians or medical workers

The invention further provides a cosmetic counseling method for supporting cosmetology, which includes providing a cosmetic method, agent for maintaining or promoting collagen productivity, composition or product of the invention to a subject.

### EXAMPLES

The present invention will now be explained in greater detail by examples. However, the invention is in no way limited by the examples.

### Material and Method

### <Preparation of Wavelength Conversion Substances>

Wavelength conversion substances were prepared in the following manner.

### (1) Zinc Oxide Phosphor

Lumate G by Sakai Chemical Industry Co., Ltd. was used. Lumate G is a zinc oxide phosphor obtained by doping ZnO with a sulfur-containing compound and then firing as described in International Patent Publication No. 2018/004006, the absorption spectrum having a peak wavelength at 365 nm and the emission spectrum having a peak wavelength at 510 nm.

### (2) Magnesium Titanate Phosphor

Lumate R by Sakai Chemical Industry Co., Ltd. was used. Lumate R is a magnesium titanate phosphor obtained by doping MgTiO₃ with manganese, the absorption spectrum having a peak wavelength at 365 nm and the emission spectrum having peak wavelengths in the range of 660 to 680 nm.

### (3) C-Phycocyanin

C-phycocyanin is obtained from Spirulina platensis extract, and the absorption spectrum has a peak wavelength at 350 nm and the emission spectrum has peak wavelengths at 640 nm and 700 nm.

Preparation method: A film containing 10% Zinc Oxide phosphor, a film containing 10% magnesium titanate phosphor were used, and C-phycocyanin was dissolved in ultrapure water at a concentration of 1% and used as a solution.
Cells: human skin-derived fibroblasts
Collagen assay kit: Cosmo Bio Co., COL-001
DNA-quantitation kit: Cosmo Bio Co., AK06
Artificial Sunlight Irradiation System: Celick Corporation: XC-500BF

### <Test Method>

### Artificial Sunlight Irradiation

Human skin-derived fibroblasts cultured in 75 cm² flasks were treated with trypsin, and after harvesting the cells, the cells were seeded at a density of 2×10⁵ cells/dish using medium on five 6-well plates according to the plate arrangement below. After three days of seeding, all five plates were washed once with PBS 2 mL, and then freshly PBS 2 mL was added and the plates were capped with a lid. Plates 2-5 covered the left half with aluminum seal. In Plate 2, a lid was further placed on the lid of the cultured 6 well plate, and in Plate 3, zinc oxide phosphor film was placed on the top of the cultured plate, and an unused lid was further placed. In Plate 4, as in Plate 2, a magnesium titanate phosphor film was placed on the lid, and a lid was further put on them. In Plate 5, a plate containing 2 mL/well of aqueous C-phycocyanin solution was placed position on top of the cultured plate with a lid uncovered. In this situation, the phosphor film did not directly contact with the cell solution, so that the artificial sunlight passed through the phosphor film was irradiated to the cell solution.

Plates 2 to 5 were placed on a heat storage material of 20° C. so as to prevent the temperature from rising due to heat, and the output knob was set to the maximum at a distance of about 70 cm from the artificial sunlight for irradiation. Irradiation time was 80 minutes. For Plate 1, irradiation was not conducted, and placed under room temperature with light shielding for 80 minutes. After the irradiation time, PBS in all plates was replaced by medium and photographed by microscope immediately after irradiation and 3 days after irradiation.

### <Collagen Quantification>

After 3 days from illumination, culture supernatants were removed from wells for collagen quantitation in each treatment group and washed with PBS. Collagen in human fibroblast layers was extracted and measured according to the protocol of the collagen quantitation kit (https://search.cosmobio.co.jp/cosmo_searchp/search_gate2/docs/CSR_/COL001.20190327.pdf). In addition, the amount of DNA in samples after collagen extraction was determined for correction of collagen quantitative results according to the protocol of the Quantitative Kit of DNA (https://search.cosmobio.co.jp/cosmo)_search_p/search_gate2/docs/PMC_/AK06.20140507.pdf).

### <Results>

UV-irradiated fibroblasts produced 14 μg collagen/well, whereas UV-irradiated fibroblasts in zinc oxide phosphor, magnesium titanate phosphor, and C-phycocyanin groups produced 15, 23, and 25 μg collagen/well, respectively.

From the above results, it was found that the wavelength conversion substance not only suppresses the decrease in collagen productivity of the cells due to UV irradiation, but also has an effect of promoting the collagen productivity of the cells by utilizing UV light. The prevention and improvement of wrinkles, photoaging, etc. are expected, when the collagen production productivity of the skin cell is maintained or promoted.

The embodiments of the invention described above are not intended to place limitations on the invention, and various modifications including cosmetics and drug compositions may be incorporated, which fall within the gist of the invention.

1. An agent for maintaining or promoting collagen productivity in cells comprising a wavelength conversion substance as an active ingredient,
wherein the wavelength conversion substance converts the wavelength of ultraviolet light contained in incident light to emit emission light having a wavelength longer than the wavelength of the ultraviolet light.

2. The agent for maintaining or promoting collagen productivity according to claim 1, wherein the ultraviolet light has a peak wavelength between 200 nm and 400 nm.

3. The agent for maintaining or promoting collagen productivity according to claim 1 or 2, wherein the emission light has a peak wavelength between 450 nm and 700 nm.

4. The agent for maintaining or promoting collagen productivity according to any one of claims 1 to 3, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, and polyphenols; and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, and meta-aminophenol.

5. The agent for maintaining or promoting collagen productivity according to claim 4, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.

6. A composition comprising the agent for maintaining or promoting collagen productivity according to any one of claims 1 to 5.

7. The composition according to claim 6, wherein the composition is a skin external composition for maintaining or promoting collagen productivity in cells under irradiation of light containing ultraviolet light.

8. A cosmetic method for maintaining or promoting collagen productivity activating skin of a subject, comprising:
applying the composition according to claim 6 or 7 to skin of a subject, and
exposing the composition-applied skin with light containing ultraviolet light.

9. A product comprising the agent for maintaining or promoting collagen productivity according to any one of claims 1 to 5.

10. The product according to claim 9, wherein the product is for maintaining or promoting collagen productivity under irradiation of light which contains ultraviolet light.

11. A cosmetic method for maintaining or promoting collagen productivity in skin cells of a subject, comprising:
bringing light containing ultraviolet light through the product according to claim 9 or 10, and
exposing the skin of the subject to light passing through the product.
